(19) United States
(12) Patent Application Publication
(10) Pub. No.: US 2017/0290749 A1
(43) Pub. Date: Oct. 12, 2017

(54) KERATIN FIBRE BLEACHING COMPOSITION

(71) Applicant: BEAUTY & BUSINESS S.p.A., Milano (IT)

(72) Inventors: Gianluca Borgnini, Carvico (BG) (IT); Francesco Facheris, Dalmine (BG) (IT); Alberto Finazzi, Bergamo (IT); Antonio Consoli, Urgnano (BG) (IT); Katiuscia Grevalcuore, Bergamo (IT)

(21) Appl. No.: 15/477,142

(22) Filed: Apr. 3, 2017

(30) Foreign Application Priority Data
Apr. 6, 2016 (IT) 102016000035302

(51) Int. Cl. A61K 8/23; A61K 8/22; A61K 8/73; A61K 8/37; A61K 8/86; A61Q 5/08; A61K 8/36

(52) U.S. Cl. CPC A61K 8/23; A61Q 5/08; A61K 8/22; A61K 8/36; A61K 8/37; A61K 8/86; A61K 8/73

(57) ABSTRACT

The invention relates to hair bleaching compositions comprising persulfate ions, alkalizing agents, high-molecular-weight polyethylene glycol (PEG), rheological agents and silicates.

This U.S. Non-Provisional Application claims priority to and the benefit from Italian Patent Application No. 102016000035302 filed on Apr. 6, 2016, the content of which is incorporated herein by reference in its entirety.

The invention relates to hair bleaching compositions comprising persulfate ions, alkalizing agents, high-molecular-weight polyethylene glycol (PEG), rheological agents and silicates.

### PRIOR ART

Hair bleaching is a process that leads to oxidation of melanin pigment and loss of the natural hair color.

Depending on the initial hair type and color, the bleaching process can lead to different degrees of lightening, according to the concentration of the active bleaching ingredients, alkaline agents, application time and temperature. Table 1 shows the starting color level (from level 1 ,the darker hair, to level 10, the lighter hair), and the bleaching capacity starting from level 4.

TABLE 1

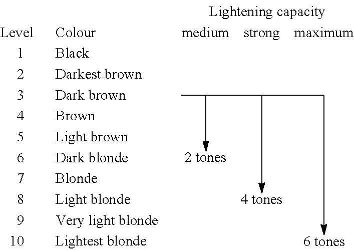

To bleach the hair by more than 4 lightening levels, products generally called bleaching powders are used in hair salons and home kits. The bleaching powder is usually mixed with an activator based on hydrogen peroxide or, occasionally, with tap water.

Bleaching pastes and creams are available in addition to powders. Said bleaching forms contain persulfates in the form of sodium, potassium or ammonium salts Ammonium salt is the most active, because it generates the ammonium ion when mixed with water or hydrogen peroxide. The ammonium ion facilitates penetration of the persulfates into the hair.

Bleaching powders that do not use ammonium persulfates but only potassium and sodium persulfates have recently widespread. Said products are known on the market as ammonia-free bleaches. Persulfates are often combined with the use of silicates and metasilicates to increase the basicity of the mixture.

Minerals such as magnesium carbonate, whose role is to balance oxygen release after dilution with the activator, are also introduced into bleaching powders.

A rheological agent such as carboxymethylcellulose or a vegetable gum is also often added. Said ingredients give the mixture of activator and bleaching powder a certain consistency so as to facilitate application with a brush, provide emollient properties, and slow down drying during the application time.

In addition to whole-head bleaching (lightening of all the hair on the head), fashion has introduced bleaching techniques that only involve bleaching complete locks or some parts thereof (e.g. meches, highlights, shatush, balayage etc.).

Hairdressers use foils to apply the bleaching product precisely. The foils serve to isolate the lock to be bleached, so that the mixture does not come into contact with nearby locks of hair during the processing time.

Bleaching techniques involving a more creative use of the bleaching mixture have become increasingly widespread in recent years, including free-hand application to obtain “artistic” results.

The problem is that when conventional bleaching powder and activator compositions are used, free-hand application is difficult because the mixture tends to lose its consistency, and precise application is also very difficult.

To prevent the product from dripping, some hairdressers use more conventional bleaching powder than activator (in a bleach-to-activator ratio of up to 2:1). Said mixtures contain high concentrations of persulfates, and involve a greater risk of damaging the hair.

Moreover, the powder and activator mixture is compact but difficult to apply, leading to the risk of obtaining an uneven result.

The purpose of the invention is to solve said problems with a novel composition, able to create a mixture suitable for free-hand application which is easy to spread, does not damage the hair, does not drip, and provides even results.

### DESCRIPTION OF THE INVENTION

It has now been found that said purposes are achieved with a composition comprising:

a) persulfate ions;

b) an alkalizing agent;

c) a high-molecular-weight polyethylene glycol (PEG) with an average degree of ethoxylation greater than or equal to 2000, and an average molecular weight greater than or equal to 100000 Da;

d) a rheological agent;

e) a clay silicate mineral.

Said composition must be mixed with an activator at the time of use.

The persulfate ions can derive from peroxydisulfate salts. The persulfate is preferably selected from sodium, potassium and ammonium persulfate or a mixture thereof.

The concentration by weight of persulfates to the total weight of the composition usually ranges between 10% and 80%, preferably between 40% and 60%.

The alkalizing agents are preferably selected from dibasic or tribasic ammonium phosphate, water-soluble silicates of alkaline or alkaline-earth metals, sodium disilicate, sodium metasilicate, dibasic or tribasic phosphates or carbonates of alkaline or alkaline-earth metals like lithium, sodium, potassium, magnesium, calcium and barium, or a mixture thereof. The alkalizing agent is preferably selected from water-soluble silicates such as sodium or potassium silicates or metasilicates or a mixture thereof.

The concentration by weight of alkalizing agent to the total weight of the composition usually ranges between 0.1% and 40%, preferably between 0.5% and 30%. The term “water-soluble silicate” relates to a silicate with a solubility in water at 25° C. greater than 0.5%, and preferably greater than 1%, by weight.

The polyethylene glycol (PEG) usable according to the invention is solid at room temperature (below 37° C.) and has an average degree of ethoxylation greater than or equal to 2000 and an average molecular weight greater than or equal to 100000 Da. The PEG preferably used has an average degree of ethoxylation of 90000 and an average molecular weight of 4000000 Da.

The concentration by weight of PEG to the total weight of the composition usually ranges between 0.01% and 20%, preferably between 0.1% and 10%.

The function of the solid PEG is to increase the viscosity of the cream obtained by mixing the bleaching powder according to the invention with an activator (usually an aqueous formulation based on hydrogen peroxide), giving it a particular “sticky consistency. This consistency allows very precise free-hand application without smears, with no need to separate the locks of hair with foils (leading to a saving of time and material), thus enabling the hairdresser to express his/her creativity to the utmost and obtain the most satisfactory results.

The use of high-molecular-weight solid PEGs is already known in the hair-styling field, for example for the formulation of modelling pastes, but its application in the field of bleaching powders is novel, and is the object of the invention. The use of low-molecular-weight PEGs (under 10000 Da) is known in the field of bleaching powders. Said PEGs, which mostly have a liquid or waxy consistency at room temperature (20-25° C.), are not used as viscosity-increasing agents but as “wetting agents”. For example, in patents U.S. Pat. No. 6,274,125 and WO2012159926, the bleaching powder “moistened” with PEG compacts to form granules or tablets while remaining solid, but less volatile (dust-free).

Polymers of natural or synthetic origin can be used as rheological agents. Examples of natural rheological agents include seaweed extracts such as alginates, carrageenans and agar; guar gum and derivatives thereof, xanthan gum, gum arabic, konjac gum, gum tragacanth, gum agar or mixtures thereof; amides, preferably modified amides; dextrins; cellulose and cellulose derivatives; pectins; chitosan and derivatives thereof; anionic polysaccharides; and soy-bean polysaccharide derivatives. Examples of synthetic polymers include crosslinked or non-crosslinked polyvinylpyrrolidone derivatives; acrylic acid polymers or salts thereof; and crosslinked or non-crosslinked polyacrylamides.

The concentration by weight of thickening agent to the total weight of the composition can range from 0.01 to 20%, preferably from 0.1 to 10%.

Kaolin, bentonite or talc is preferably used as silicate mineral of natural origin. The concentration by weight of clay silicate mineral to the total weight of the composition usually ranges between 1% and 40%, preferably between 5% and 30%.

The composition can also include a pH adjuster selected, for example, from urea, allantoin, arginine, tripotassium phosphate, sodium saccharine, citric acid and phosphoric acid or combinations thereof.

The quantity of pH adjuster can range between 0.1 and 20% by weight, preferably between 0.2 and 10% by weight.

The composition can also contain minerals such as hydrophobic or hydrophilic silica, clay, ceramic beads, calcium carbonate, titanium oxide, magnesium oxides, aluminium silicates and derivatives thereof, nylon, vinylidene chloride/acrylonitrile/methacrylonitrile copolymer microbeads, and micronised or non-micronised vegetable powder.

Among the mentioned silica, hydrophilic fumed silica (Aerosil® 90, 130, 150, 200, 300 and 380, Degussa Huls) can be used in particular.

The quantity of said ingredients can range between 0.1 and 20% by weight, preferably between 0.2 and 10% by weight.

The compositions can also contain pigments, such as iron oxides, titanium oxides, zinc oxides, chromium oxides, borosilicates, bismuth oxychloride, aluminium hydroxide lakes (FD&C Lakes or D&C colors), the ultramarine dye family, manganese violet, or ferric ferrocyanide. Other particular pigments which can be used are marketed under the names WATERSPERSE® (S.A COLOR); UNIPURE (SENSIENT); CELLINI® (BASF); DISTINCTIVE® (RESOURCE OF NATURE), COLORONA® MERCK and WD (DAITO KASEI).

The concentration by weight of the pigments to the total weight of the composition can range from 0.01 to 10%, preferably from 3 to 8%.

The compositions can also contain oxidation dyes (or salts thereof), such as those with the following INCI names (European Community Decision 2006/257/EC, as amended):

1-Acetoxy-2-Methylnaphthalene, 5-Amino-4-Chloro-o-Cresol, 4-Amino-m-Cresol, 6-Amino-m-Cresol, 3-Amino-2,4-Dichlorophenol, 6-Amino-2,4-Dichloro-m-Cresol, 3-Amino-2,4-Dichlorophenol, 5-Amino-2,6-Dimethoxy-3-Hydroxypyridine, 5-Amino-2,6-Dimethoxy-3-Hydroxypyridine, 3-Amino-2,6-Dimethylphenol, 2-Amino-5-Ethylphenol, 5-Amino-4-Fluoro-2-Methylphenol Sulfate, 2-Amino-4-Hydroxyethylaminoanisole, 2-Amino-4-Hydroxyethylaminoanisole, 2-Amino-3-Hydroxypyridine, 4-Amino-2-Hydroxytoluene, 2-Aminomethyl-p-Aminophenol, 4-Amino-2-Nitrodiphenylamine-2′-Carboxylic Acid, m-Aminophenol, o-Aminophenol, p-Aminophenol, 1,3-Bis-(2,4-Diaminophenoxy)propane, 4,6-Bis(2-Hydroxyethoxy)-m-Phenylenediamine, 2,6-Bis(2-Hydroxyethoxy)-3,5-Pyridinediamine, N,N-Bis(2-Hydroxyethyl)-p-Phenylenediamine, 4-Chloro-2-Aminophenol, 2-Chloro-p-Phenylenediamine, 4-Chlororesorcinol, N-Cyclopentyl-m-Aminophenol, 3,4-Diaminobenzoic Acid, 4,5-Diamino-1-((4-Chlorophenyl)Methyl)-1H-Pyrazole-Sulfate, 2,3-Diaminodihydropyrazolo Pyrazolone Dimethosulfonate, 2,4-Diaminodiphenylamine, 4,4′-Diaminodiphenylamine, 2,4-Diamino-5-Methylphenetole, 2,4-Diamino-5-Methylphenoxyethanol, 4,5-Diamino-1-Methylpyrazole, 2,4-Diaminophenol 2,4-Diaminophenoxyethanol, 2,6-Diaminopyridine, 2,6-Diamino-3((Pyridin-3-yl)Azo)Pyridine, N,N-Diethyl-m-Aminophenol, N,N-Diethyl-p-Phenylenediamine, N,N-Diethyltoluene-2,5-Diamine, 2,6-Dihydroxy-3,4-Dimethylpyridine, 2,6-Dihydroxyethylaminotoluene, Dihydroxyindole, Dihydroxyindoline, 2,6-Dimethoxy-3,5-Pyridinediamine, m-Dimethylaminophenyl Urea, N,N-Dimethyl-p-Phenylenediamine,2,6-Dimethyl-p-Phenylenediamine, N,N-Dimethyl-2,6-Pyridinediamine, 4-Ethoxy-m-Phenylenediamine, 3-Ethylamino-p-Cresol, 4-Fluoro-6-Methyl-m-Phenylenediamine, 1-Hexyl 4,5-Diamino Pyrazole Sulfate, Hydroquinone, Hydroxyanthraquinoneaminopropyl Methyl Morpholinium Methosulfate, Hydroxybenzomorpholine, Hydroxyethoxy Aminopyrazolopyridine, Hydroxyethylaminomethyl-p-Aminophenol, 1-Hydroxyethyl 4,5-Diamino-Pyrazole, Hydroxyethyl-2,6-Dinitro-p-Anisidine, Hydroxyethyl-3,4-Methylenedioxyaniline, Hydroxyethyl-p-Phenylenediamine, 2-Hydroxyethyl Picramic Acid, 6-Hydroxyindole, Hydroxypropyl Bis(N-Hydroxyethyl-p-Phenylenediamine), Hydroxypropyl-p-Phenylenediamine, Hydroxypyridinone, Isatin, N-Isopropyl 4,5-Diamino Pyrazole, N-Methoxyethyl-p-Phenylenediamine, 6-Methoxy-2-methylamino-3-aminopyridine, 2-Methoxymethyl-p-Aminophenol, 2-Methoxymethyl-p-Phenylenediamine, 2-Methoxy-p-Phenylenediamine, 6-Methoxy-2,3-Pyridinediamine, 4-Methoxytoluene-2,5-Diamine, p-Methylaminophenol, 4-Methylbenzyl 4,5-Diamino Pyrazole, 2,2′-Methylenebis 4-Aminophenol, 3,4-Methylenedioxyaniline, 3,4-Methylenedioxyphenol, 2-Methyl-5-Hydroxyethylaminophenol, Methylimidazoliumpropyl p-Phenylenediamine, 2-Methyl-1-Naphthol, 2-Methylresorcinol, 1,5-Naphthalenediol, 1,7-Naphthalenediol, 2,3-Naphthalenediol, 2,7-Naphthalenediol, 1-Naphthol, 2-Naphthol, PEG-3 2,2′-Di-p-Phenylenediamine, p-Phenetidine, m-Phenylenediamine, p-Phenylenediamine, Phenyl Methyl Pyrazolone, N-Phenyl-p-Phenylenediamine, Picramic Acid, Pyrocatechol, Pyrogallol, Resorcinol, Sodium Picramate, Tetraaminopyrimidine, Tetrahydro-6-Nitroquinoxaline, Tetrahydropyranyl, Resorcinol, Toluene-2,5-Diamine Toluene-2,6-Diamine, Toluene-3,4-Diamine, 2,5,6-Triamino-4-Pyrimidinol, 1,2,4-Trihydroxybenzene.

The total quantity of the oxidation dyes in the colouring preparation according to the invention preferably ranges between about 0.001 and 20% by weight, more preferably between about 0.002 and 10% by weight, and even more preferably between about 0.01 and 6.0% by weight.

The compositions can also contain direct dyes, such as those with the following INCI names (European Community Decision 2006/257/EC, as amended):

Acid green 25, Acid blue 74, Acid blue 3, Acid blue 9, Acid red 18, Acid red 184, Acid red 195, Acid red 27, Acid red 33, Acid red 35, Acid red 51, Acid red 73, Acid red 87, Acid red 92, Acid red 95, Acid violet 43, Acid violet 9, Acid yellow 23, Acid yellow 3, Acid yellow 36, Acid yellow 73, Acid orange 6, Acid orange 7, Acid green 1, Acid green 50, Acid Blue 1, Acid Blue 62, Acid Brown 13, Acid Orange 3, Acid Orange 24, Acid Orange 7, Acid Red 14, Acid Red 35, Acid Red 52, Acid Yellow 1, 2-Amino-6-Chloro-4-Nitrophenol, 4-Amino-2-Nitrodiphenylamine-2′-Carboxylic Acid, 2-Amino-3-Nitrophenol, 2-Amino-4-Nitrophenol, 2-Amino-5-Nitrophenol, 4-Amino-2-Nitrophenol, 4-Amino-3-Nitrophenol, Basic Blue 3, Basic Blue 7, Basic Blue 9, Basic Blue 26, Basic Blue 47, Basic Blue 75, Basic Blue 99, Basic Blue 124, Basic Brown 4, Basic Brown 16, Basic Brown 17, Basic Green 1, Basic Green 4, Basic Orange 1, Basic Orange 2, Basic Orange 31, Basic Red 1, Basic Red 1:1, Basic Red 2, Basic Red 22, Basic Red 46, Basic Red 51, Basic Red 76, Basic Red 118, Basic Violet 1, Basic Violet 2, Basic Violet 3, Basic Violet 4, Basic Violet 10, Basic Violet 11:1, Basic Violet 14, Basic Violet 16, Basic Yellow 28, Basic Yellow 40, Basic Yellow 57, Basic Yellow 87, N,N′-Bis(2-Hydroxyethyl)-2-Nitro-p-Phenylenediamine, 2-Chloro-6-Ethylamino-4-Nitrophenol, 2-Chloro-5-Nitro-N-Hydroxyethyl p-Phenylenediamine, N,N′-Dimethyl-N-Hydroxyethyl-3-Nitro-p-Phenylenediamine, Direct Black 51, Direct Red 23, Direct Red 80, Direct Red 81, Direct Violet 48, Direct Yellow 12, Disperse Black 9, Disperse Blue 1, Disperse Blue 3, Disperse Blue 7, Disperse Blue 377, Disperse Brown 1, Disperse Orange 3, Disperse Red 11, Disperse Red 15, Disperse Red 17, Disperse Violet 1, Disperse Violet 4, Disperse Violet 15, HC Blue No. 2, HC Blue No. 4, HC Blue No. 5, HC Blue No. 6, HC Blue No. 8, HC Blue No. 9, HC Blue No. 10, HC Blue No. 11, HC Blue No. 12, HC Blue No. 13, HC Blue No. 14, HC Blue No. 15, HC Blue No.16, HC Blue No. 17, HC Blue No. 18, HC Brown No. 1, HC Brown No. 2, HC Green No. 1, HC Orange No. 1, HC Orange No. 2, HC Orange No. 3, HC Orange No. 5, HC Orange No. 6, HC Red No. 1, HC Red No. 3, HC Red No. 7, HC Red No. 8, HC Red No. 9, HC Red No. 10, HC Red No. 11, HC Red No. 13, HC Red No. 14, HC Red No. 15, HC Red No. 17, HC Red No. 18, HC Violet No. 1, HC Violet No. 2, HC Yellow No. 2, HC Yellow No. 4, HC Yellow No. 5, HC Yellow No. 6, HC Yellow No. 7, HC Yellow No. 8, HC Yellow No. 9, HC Yellow No. 10, HC Yellow No. 11, HC Yellow No. 12, HC Yellow No. 13, HC Yellow No. 14, HC Yellow No. 15, HC Yellow No. 16, HC Yellow No. 17, 2-Hydroxyethylamino-5-Nitroanisole, Hydroxyethyl-2-Nitro-p-Toluidine, 4-Hydroxypropylamino-3-Nitrophenol, 3-Methylamino-4-Nitrophenoxyethanol, 3-Nitro-4-Aminophenoxyethanol, 3-Nitro-p-Cresol, 2-Nitro-5-Glyceryl Methylaniline, 4-Nitroguaiacol, 3-Nitro-p-Hydroxyethylaminophenol, 2-Nitro-N-Hydroxyethyl-p-Anisidine, Nitrophenol, 4-Nitrophenyl Aminoethylurea, 4-Nitro-o-Phenylenediamine, 4-Nitro-m-Phenylenediamine, 4-Nitro-o-Phenylenediamine, 2-Nitro-p-Phenylenediamine, 6-Nitro-2,5-Pyridinediamine, 6-Nitro-o-Toluidine, Pigment Blue 15, Pigment Blue 15:1, Pigment Violet 23, Pigment Yellow 13, Solvent Black 3, Solvent Black 5, Solvent Blue 35, Solvent Yellow 85, Solvent Yellow 172, Tetrabromophenol Blue, Tetrahydro-6-Nitroquinoxaline, Tetrahydropyranyl Resorcinol.

Other dyes which can be used are those described in WO 2014202152.

The total quantity of direct dye in the colouring preparation according to the invention preferably ranges between about 0.001 and 20% by weight, more preferably between about 0.002 and 10% by weight, and even more preferably between about 0.01 and 6.0% by weight.

Examples of natural direct dyes include those based on lawsone, juglone, alizarin, purpurin, carminic acid, kermesic acid, purpurogallin, protocatechualdehyde, indigo, isatin, curcumin, spinulosine and apigenidine. Extracts or decoctions containing said natural dyes and, in particular, henna-based packs or extracts, can also be used.

The composition described must be mixed with an activator shortly before application to the hair. When the bleaching composition is mixed with the activator, which in most cases is acidic (pH about 2 to 6.5), the pH of the ready-to-use composition according to the invention acquires a value determined by the quantity of alkali in the bleaching composition and the quantity of acid in the activator, and by the mixing ratio. Depending on the composition, the ready-to-use bleaches can be weakly acid, neutral or alkaline, and have a pH ranging from about 3 to 11, preferably from 6.5 to 11.

“Activator” means water, hydrogen peroxide, carbamide peroxide, perborates and persulfates or peracids, preferably hydrogen peroxide. The quantity can range from 0.1 to 50% by weight.

The activating solution of hydrogen peroxide can optionally contain 0.01 to 10%, preferably 0.05 to 5% by weight, of one or more polymers selected from: Acrylates Copolymer, Acrylates/Steareth-20 Methacrylate Copolymer, Acrylates/Vinyl Neodecanoate Crosspolymer, Acrylates/Methacrylamide Copolymer, Polyacrylamide, Sodium Polyacrylate, Sodium Acrylate/Acryloyldimethyltaurate/Dimethylacrylamide Crosspolymer, sodium acrylates crosspolymer-2, Methoxy PEG-17/Methoxy PEG-11/HDI Isocyanurate Trimer Crosspolymer, Polyurethane-39, PEG-150/Stearyl Alcohol/SMDI Copolymer, Polyquaternium-37, Polyacrylate Crosspolymer-11, Sodium Acryloyldimethyltaurate/VP Crosspolymer, Ammonium Acryloyldimethyltaurate/VP Copolymer, Ammonium Acryloyldimethyltaurate/Beheneth-25 Methacrylate Crosspolymer, Polyacrylate crosspolymer-6, Hydroxyethyl Acrylate/Sodium Acryoyldimethyltaurate/Steareth-20 Methacrylate Copolymer, Sodium Acrylate/Sodium Acryloyldimethyl Taurate Copolymer, Acrylates/Beheneth-25 Methacrylate Copolymer. The amount of the mixture of bleaching composition and activator to be applied depends on the amount of hair to be bleached, usually about 5 to 200 grams. The ready-to-use bleaching mixture is left on the hair for 2 to 90 minutes at the temperature of 5 to 50° C., preferably for 45 minutes at 30° C.; the hair is then rinsed with water and dried. If necessary, the hair is washed with shampoo after rinsing and optionally rinsed again with a weak organic acid, such as an aqueous solution of tartaric acid. The hair is then dried.

### EXAMPLES

The ingredients listed in the examples are named according to the INCI nomenclature, as defined above. Table 2 describes the compositions of the hydrogen peroxide-based activators used for the examples, indicated with the various strengths (A: 40 volumes, B: 30 volumes, C: 20 volumes, D: 10 volumes).

TABLE 2
Activators

| | A | B | C | D |
|---|---|---|---|---|
| INGREDIENTS | % | % | % | % |
| AQUA (WATER) | q.s. to 100 | q.s. to 100 | q.s. to 100 | q.s. to 100 |
| HYDROGEN | 12 | 9 | 6 | 3 |
| PEROXIDE | | | | |
| CETEARYL ALCOHOL | 3 | 3 | 3 | 3 |
| CETEARETH-20 | 0.6 | 0.6 | 0.6 | 0.6 |
| PHOSPHORIC ACID | 0.1 | 0.1 | 0.1 | 0.1 |
| SODIUM STANNATE | 0.2 | 0.2 | 0.2 | 0.2 |
| SODIUM LAURETH | 0.1 | 0.1 | 0.1 | 0.1 |
| SULFATE | | | | |
| PROPYLENE GLYCOL | 0.1 | 0.1 | 0.1 | 0.1 |
| DISODIUM | 0 | 0 | 0 | 0 |
| PYROPHOSPHATE | | | | |
| DIMETHICONE | 0.1 | 0.1 | 0.1 | 0.1 |
| PEG-40 CASTOR OIL | 0.1 | 0.1 | 0.1 | 0.1 |
| PENTASODIUM | 0.1 | 0.1 | 0.1 | 0.1 |
| PENTETATE | | | | |
| ETIDRONIC ACID | 0.1 | 0.1 | 0.1 | 0.1 |
| C12-13 ALKYL | 1 | 1 | 1 | 1 |
| LACTATE | | | | |

Composition E in Table 3 is the bleaching composition according to the invention, while composition F is conventional.

TABLE 3
Bleaching composition

| | COMPOSITION: | |
|---|---|---|
| | E* | F |
| INGREDIENT NAME (INCI) | w/w % | w/w % |
| SODIUM SILICATE | 20 | 20 |
| AMMONIUM PERSULFATE | 25 | 25 |
| KAOLIN | 20 | 6 |
| POTASSIUM PERSULFATE | 10 | 10 |
| PARAFFINUM LIQUIDUM (MINERAL OIL) | 3 | 3 |
| SODIUM STEARATE | 3 | 3 |
| MAGNESIUM CARBONATE | 13 | 30 |
| XANTHAN GUM | — | 1 |
| CYAMOPSIS TETRAGONOLOBA GUM | 2.5 | — |
| (CYAMOPSIS TETRAGONOLOBA (GUAR) GUM) | | |
| DISODIUM EDTA | 1 | 1 |
| GUAR HYDROXYPROPYLTRIMONIUM | 1 | — |
| CHLORIDE | | |
| SILICA | 1 | 1 |
| PEG-90M | 0.5 | — |

Compositions E and F were applied without foils by an experienced hairdresser to 3 models, and the following parameters were evaluated:

FREE-HAND APPLICABILITY: drips or does not drip.

EASE OF FREE-HAND APPLICATION: excellent, good, fairly good, fair or poor.

TENDENCY TO STAIN HAIR CLOSE TO APPLICATION: does not stain, stains, stains badly.

EVENNESS OF COLOUR AFTER RINSING: high, normal, very poor.

A 50 g dilution of composition E or F with 75 g of activator A was used for model 1, and a 50 g dilution of composition E or F with 100 g of activator B for model 2. A 50 g dilution of composition E with 100 g of activator C and a 40 g dilution of composition F with 10 g of activator C were used for model 3.

TABLE 4
Result of application of compositions E and F to the 3 models

| | Model 1 | | Model 2 | | Model 3 | |
|---|---|---|---|---|---|---|
| | E | F | E | F | E | F |
| FREE-HAND | No | Dripping | No | Dripping | No | No |
| APPLICABILITY | dripping | | dripping | | dripping | dripping |
| EASE OF FREE- | Excellent | Very | Good | Very | Good | Poor |
| HAND | | good | | good | | |
| APPLICATION | | | | | | |
| TENDENCY TO | No | Staining | No | Badly | No | No |
| STAIN HAIR | staining | | staining | staining | staining | staining |
| CLOSE TO | | | | | | |
| APPLICATION | | | | | | |
| EVENNESS OF | High | Normal | High | Normal | High | Very |
| COLOUR AFTER | | | | | | Poor |
| RINSING | | | | | | |

Conclusion: the composition according to the invention has the better performance in a free-hand application method

TABLE 5
Other Compositions according to the invention

| COMPOSITION: | G | H | I | L | M | N | O | P | Q | R |
|---|---|---|---|---|---|---|---|---|---|---|
| INGREDIENT NAME (INCI) | w/w % | w/w % | w/w % | w/w % | w/w % | w/w % | w/w % | w/w % | w/w % | w/w % |
| SODIUM SILICATE | 10 | 30 | 25 | 20 | 20 | 20 | 20 | 20 | 20 | 10 |
| SODIUM METASILICATE | 5 | 3 | — | — | — | — | 5 | — | — | — |
| AMMONIUM PERSULFATE | 20 | — | 25 | 25 | 25 | 25 | 25 | 25 | 25 | 20 |
| KAOLIN | 10 | 10 | 9 | 15 | 20 | 15 | 7 | 8 | 10 | 40 |
| POTASSIUM PERSULFATE | 10 | 10 | 10 | 10 | 10 | 10 | 10 | 10 | 10 | 10 |
| PARAFFINUM LIQUIDUM (MINERAL OIL) | 4 | 4 | 2 | 1 | 4 | 3 | 2 | 4 | 4 | 4 |
| SODIUM STEARATE | 1 | 2 | 3 | 4 | 2 | 6 | 1 | 3 | 3 | 3 |
| CYAMOPSIS TETRAGONOLOBA GUM | 2.5 | 3 | 2 | 3 | 3 | 2.5 | 3 | 3 | 1 | 1 |
| (CYAMOPSIS TETRAGONOLOBA (GUAR) GUM) | | | | | | | | | | |
| DISODIUM EDTA | 1 | 1 | 1 | 1 | 1 | 1 | 1 | 1 | 1 | 1 |
| GUAR HYDROXYPROPYLTRIMONIUM | 1 | 1 | 1.5 | 1 | 1.5 | 1 | 2 | 1.5 | 1 | 1 |
| CHLORIDE | | | | | | | | | | |
| SILICA | 1 | 2 | 0.5 | 1 | 3 | 1.5 | 1.5 | 1 | 0.5 | 0.5 |
| PEG-90M | 0.5 | 1 | 0.3 | 2 | 3 | 0.7 | 0.8 | 1 | 0.6 | 0.6 |
| PARFUM | 0.5 | 0.5 | 0.5 | 0.5 | 0.5 | 0.5 | 0.5 | 0.5 | 0.5 | 0.5 |
| ACID RED 92 | — | — | 0.5 | — | — | — | — | — | — | — |
| MAGNESIUM CARBONATE | 29.2 | 30 | 19.4 | 14 | 6 | 10 | 15 | 15 | 20 | 5 |
| DIMETHICONE | 0.3 | — | — | — | — | — | — | — | — | — |
| XANTHAN GUM | — | — | — | 0.5 | — | — | — | — | — | — |
| HYDROXYETHYLCELLULOSE | — | — | — | — | 0.3 | — | — | — | — | — |
| MAGNESIUM OXIDE | — | — | 0.3 | — | — | — | — | — | — | — |
| TETRABROMOPHENOL BLUE | — | — | — | — | — | — | 1 | — | — | — |
| TITANIUM DIOXIDE | 3 | — | — | — | — | 0.2 | — | — | — | — |
| BASIC YELLOW 87 | — | — | — | — | — | 0.8 | — | — | — | — |
| CI 42520 | — | — | — | 0.5 | — | — | — | — | — | — |
| CI 77007 | — | — | — | 0.5 | — | — | — | — | — | — |
| ACID ORANGE 7 | — | — | — | — | 0.5 | — | — | — | — | — |
| HC BLUE 15 | — | — | — | — | — | 0.4 | — | — | — | — |
| ACID YELLOW 1 | — | — | — | 0.3 | — | — | — | — | — | — |
| BASIC RED 51 | — | — | — | — | — | 1 | — | — | — | — |
| AMMONIUM CHLORIDE | | 2.5 | | 0.7 | 0.2 | 1.4 | 5.2 | 4 | 2.4 | 2.4 |
| SODIUM LAURYL SULFATE | 1 | | | | | | | 1 | 1 | 1 |
| HC BLUE 16 | — | — | — | — | — | — | — | 2 | | |

TABLE 6
Other Activators according to the invention

| | S | T | U | V | Z | AA | AB | AC | AD | AE | AF | AG | AH | AI | AL | AM | AN | AO | AP |
|---|---|---|---|---|---|---|---|---|---|---|---|---|---|---|---|---|---|---|---|
| INGREDIENTS | % | % | % | % | % | % | % | % | % | % | % | % | % | % | % | % | % | % | % |
| AQUA (WATER) | q.s. to | q.s. to | q.s. to | q.s. to | q.s. to | q.s. to | q.s. to | q.s. to | q.s. to | q.s. to | q.s. to | q.s. to | q.s. to | q.s. to | q.s. to | q.s. to | q.s. to | q.s. to | q.s. to |
| | 100 | 100 | 100 | 100 | 100 | 100 | 100 | 100 | 100 | 100 | 100 | 100 | 100 | 100 | 100 | 100 | 100 | 100 | 100 |
| HYDROGEN PEROXIDE | 1 to 12 | 1 to 12 | 1 to 12 | 1 to 12 | 1 to 12 | 1 to 12 | 1 to 12 | 1 to 12 | 1 to 12 | 1 to 12 | 1 to 12 | 1 to 12 | 1 to 12 | 1 to 12 | 1 to 12 | 1 to 12 | 1 to 12 | 1 to 12 | 1 to 12 |
| CETEARYL ALCOHOL | 0.5 | 2 | — | 1 | — | 5 | — | — | — | — | — | — | — | 3 | 3 | 3 | 3 | 3 | 3 |
| CETEARETH-20 | 0.6 | 0.6 | — | 0.6 | — | 0.6 | — | — | — | — | — | — | — | 0.6 | 0.6 | 0.6 | 0.6 | 0.6 | 0.6 |
| PHOSPHORIC ACID | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 |
| SODIUM STANNATE | 0.2 | 0.2 | 0.2 | 0.2 | 0.2 | 0.2 | 0.2 | 0.2 | 0.2 | 0.2 | 0.2 | 0.2 | 0.2 | 0.2 | 0.2 | 0.2 | 0.2 | 0.2 | 0.2 |
| SODIUM LAURETH | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 |
| SULFATE | | | | | | | | | | | | | | | | | | | |
| PROPYLENE GLYCOL | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 |
| DISODIUM | 0 | 0 | 0 | 0 | 0 | 0 | 0 | 0 | 0 | 0 | 0 | 0 | 0 | 0 | 0 | 0 | 0 | 0 | 0 |
| PYROPHOSPHATE | | | | | | | | | | | | | | | | | | | |
| DIMETHICONE | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 |
| PEG-40 CASTOR OIL | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 |
| PENTASODIUM | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 |
| PENTETATE | | | | | | | | | | | | | | | | | | | |
| EDTA | 0.05 | 0.05 | 0.05 | 0.05 | 0.05 | 0.05 | 0.05 | 0.05 | 0.05 | 0.05 | 0.05 | 0.05 | 0.05 | 0.05 | 0.05 | 0.05 | 0.05 | 0.05 | 0.05 |
| ETIDRONIC ACID | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 | 0.1 |
| C12-13 ALKYL LACTATE | 1 | 1 | 1 | 1 | 1 | 1 | 1 | 1 | 1 | 1 | 1 | 1 | 1 | 1 | 1 | 1 | 1 | 1 | 1 |
| ACRYLATES COPOLYMER | 0.5 | — | — | — | — | — | — | — | — | — | — | — | — | — | — | — | — | — | — |
| ACRYLATES/STEARETH-20 | — | 0.5 | — | — | — | — | — | — | — | — | — | — | 0.1 | — | — | — | — | — | — |
| METHACRYLATE | | | | | | | | | | | | | | | | | | | |
| COPOLYMER | | | | | | | | | | | | | | | | | | | |
| ACRYLATES/VINYL | — | — | 0.5 | — | — | — | — | — | — | — | — | — | — | — | — | — | — | — | — |
| NEODECANOATE | | | | | | | | | | | | | | | | | | | |
| CROSSPOLYMER | | | | | | | | | | | | | | | | | | | |
| ACRYLATES/ | — | — | — | 0.5 | — | — | — | — | — | — | — | — | — | — | — | — | — | — | — |
| METHACRYLAMIDE | | | | | | | | | | | | | | | | | | | |
| COPOLYMER | | | | | | | | | | | | | | | | | | | |
| POLYACRYLAMIDE | — | — | — | — | 0.5 | — | — | — | — | — | — | — | — | — | — | — | — | — | — |
| SODIUM POLYACRYLATE | — | — | — | — | — | 0.5 | — | — | — | — | — | — | — | — | — | — | — | — | — |
| SODIUM ACRYLATE/ | — | — | — | — | — | — | 0.5 | — | — | — | — | — | — | — | — | — | — | — | — |
| ACRYLOYLDIMETHYL- | | | | | | | | | | | | | | | | | | | |
| TAURATE/ | | | | | | | | | | | | | | | | | | | |
| DIMETHYLACRYLAMIDE | | | | | | | | | | | | | | | | | | | |
| CROSSPOLYMER | | | | | | | | | | | | | | | | | | | |
| SODIUM ACRYLATES | — | — | — | — | — | — | — | 0.5 | — | — | — | — | — | — | — | — | — | — | — |
| CROSSPOLYMER-2 | | | | | | | | | | | | | | | | | | | |
| METHOXY PEG-17/ | — | — | — | — | — | — | — | — | 0.5 | — | — | — | — | — | — | — | — | — | — |
| METHOXY PEG-11/HDI | | | | | | | | | | | | | | | | | | | |
| ISOCYANURATE | | | | | | | | | | | | | | | | | | | |
| TRIMER CROSSPOLYMER | | | | | | | | | | | | | | | | | | | |
| POLYURETHANE-39 | — | — | — | — | — | — | — | — | — | 0.5 | — | — | — | — | — | — | — | — | — |
| PEG-150/STEARYL | — | — | — | — | — | — | — | — | — | — | 0.5 | — | — | — | — | — | — | — | — |
| ALCOHOL/ | | | | | | | | | | | | | | | | | | | |
| SMDI COPOLYMER | | | | | | | | | | | | | | | | | | | |
| POLYQUATERNIUM-37 | — | — | — | — | — | — | — | — | — | — | — | 0.5 | — | — | — | — | — | — | — |
| POLYACRYLATE | — | — | — | — | — | — | — | — | — | — | — | — | 0.5 | — | — | — | — | — | — |
| CROSSPOLYMER-11 | | | | | | | | | | | | | | | | | | | |
| SODIUM OR AMMONIUM | — | — | — | — | — | — | — | — | — | — | — | — | — | — | 1 | — | — | — | — |
| ACRYLOYLDIMETHYL- | | | | | | | | | | | | | | | | | | | |
| TAURATE/VP | | | | | | | | | | | | | | | | | | | |
| CROSSPOLYMER | | | | | | | | | | | | | | | | | | | |
| AMMONIUM | — | — | — | — | — | — | — | — | — | — | — | — | — | — | — | 0.5 | — | — | — |
| ACRYLOYLDIMETHYL- | | | | | | | | | | | | | | | | | | | |
| TAURATE/BEHENETH- | | | | | | | | | | | | | | | | | | | |
| 25 METHACRYLATE | | | | | | | | | | | | | | | | | | | |
| CROSSPOLYMER | | | | | | | | | | | | | | | | | | | |
| POLYACRYLATE | — | — | — | — | — | — | — | — | — | — | — | — | — | — | — | — | 0.5 | — | — |
| CROSSPOLYMER-6 | | | | | | | | | | | | | | | | | | | |
| HYDROXYETHYL | — | — | — | — | — | — | — | — | — | — | — | — | — | — | — | — | — | 0.6 | — |
| ACRYLATE/SODIUM | | | | | | | | | | | | | | | | | | | |
| ACRYOYLDIMETHYL- | | | | | | | | | | | | | | | | | | | |
| TAURATE/ | | | | | | | | | | | | | | | | | | | |
| STEARETH-20 | | | | | | | | | | | | | | | | | | | |
| METHACRYLATE | | | | | | | | | | | | | | | | | | | |
| COPOLYMER | | | | | | | | | | | | | | | | | | | |
| SODIUM ACRYLATE/ | — | — | — | — | — | — | — | — | — | — | — | — | — | — | — | — | — | — | 0.3 |
| SODIUM | | | | | | | | | | | | | | | | | | | |
| ACRYLOYLDIMETHYL | | | | | | | | | | | | | | | | | | | |
| TAURATE COPOLYMER | | | | | | | | | | | | | | | | | | | |
| ACRYLATES/BEHENETH-25 | — | — | — | — | — | — | — | — | — | — | — | — | — | 1 | — | — | — | — | — |
| METHACRYLATE | | | | | | | | | | | | | | | | | | | |
| COPOLYMER | | | | | | | | | | | | | | | | | | | |

The compositions according to the invention illustrated in Table 5 can be mixed in dilution ratios ranging from 1:0.1 to 1:10 with the activators listed in Tables 2 and 6, or with water. Some compositions bleach and color the hair simultaneously.

1. A hair bleaching composition comprising:
a) persulphate;
b) an alkalizing agent;
c) a high molecular weight polyethylene glycol (PEG) with an average degree of ethoxylation greater than or equal to 2000, and an average molecular weight greater than or equal to 100000 Da;
d) a rheological agent;
e) a silicate mineral.

2. A composition according to claim 1, further comprising oxidation dyes and/or direct dyes.

3. A composition according to claim 1 wherein the polyethylene glycol has an average degree of ethoxylation of 90000 and an average molecular weight of 4000000 Da.

4. A composition according to claim 1 wherein the concentration by weight of polyethylene glycol compared with the total composition weight ranges from 0.01% to 20%.

5. A composition according to claim 1 wherein the persulphate is selected from sodium, potassium or ammonium persulphate or a mixture thereof.

6. A composition according to claim 1 wherein the concentration by weight of persulphate compared with the total composition weight ranges from 10% to 80%.

7. A composition according to claim 1 wherein the alkalizing agent is selected from dibasic or tribasic ammonium phosphate, water-soluble silicates of alkali metals or alkaline-earth metals, sodium disilicate, sodium metasilicate, dibasic or tribasic phosphates or carbonates of alkali metals or alkaline-earth metals and a mixture thereof.

8. A composition according to claim 1 wherein the rheological agent is selected from alginates, carrageenans, agar agar; guar gum and derivatives, xanthan gum, gum arabic, konjac gum, gum tragacanth, agar gum or mixtures thereof; amides; dextrins; cellulose and cellulose derivatives; pectins; chitosan and derivatives; anionic polysaccharides; soy-bean polysaccharide derivatives, crosslinked or non-crosslinked polyvinylpyrrolidone derivatives; acrylic acid polymers or salts thereof; and crosslinked or non-crosslinked polyacrylamides.

9. A composition according to claim 1 wherein the silicate mineral is selected from kaolin, bentonite and talc.

10. A composition according to claim 1 further comprising one or more pH correctors, hydrophobic or hydrophilic silica, clay, ceramic beads, calcium carbonate, titanium oxide, magnesium oxides, aluminium silicates and derivatives, nylon, vinylidene chloride/acrylonitrile/methacrylonitrile copolymer microbeads, micronised or non-micronised vegetable powder, pigments.

11. A composition according to claim 1 which, at the time of use, is mixed with an activator selected from water, hydrogen peroxide, carbamide peroxide, perborates, persulphates and peracids, in amounts ranging from 0.1 to 50% by weight.

12. A composition according to claim 11 wherein the activator is a solution of hydrogen peroxide optionally containing 0.01 to 10% by weight of one or more polymers selected from: Acrylates Copolymer, Acrylates/Steareth-20 Methacrylate Copolymer, Acrylates/Vinyl Neodecanoate Crosspolymer, Acrylates/Methacrylamide Copolymer, Polyacrylamide, Sodium Polyacrylate, Sodium Acrylate/Acryloyldimethyltaurate/Dimethylacrylamide Crosspolymer, sodium acrylates crosspolymer-2, Methoxy PEG-17/Methoxy PEG-11/HDI Isocyanurate Trimer Crosspolymer, Polyurethane-39, PEG-150/Stearyl Alcohol/SMDI Copolymer, Polyquaternium-37, Polyacrylate Crosspolymer-11, Sodium Acryloyldimethyltaurate/VP Crosspolymer, Ammonium Acryloyldimethyltaurate/VP Copolymer, Ammonium Acryloyldimethyltaurate/Beheneth-25 Methacrylate Crosspolymer, Polyacrylate crosspolymer-6, Hydroxyethyl Acrylate/Sodium Acryoyldimethyltaurate/Steareth-20 Methacrylate Copolymer, Sodium Acrylate/Sodium Acryloyldimethyl Taurate Copolymer, and Acrylates/Beheneth-25 Methacrylate Copolymer.
